(19) United States
(12) Patent Application Publication
(10) Pub. No.: US 2023/0414878 A1
(43) Pub. Date: Dec. 28, 2023

(54) FORMULATIONS, METHODS, AND PRE-FILLED INJECTION DEVICES WITHOUT FATTY ACID PARTICLES

(71) Applicant: W. L. Gore & Associates, Inc., Newark, DE (US)

(72) Inventors: Edward C. Gunzel, Oxford, PA (US); Thomas W. Patapoff, Captain Cook, HI (US)

(21) Appl. No.: 18/037,139

(22) PCT Filed: Nov. 15, 2021
(86) PCT No.: PCT/US2021/072413

§ 371 Date: May 16, 2023

Related U.S. Application Data
(60) Provisional application No. 63/114,192, filed on Nov. 16, 2020.

(51) Int. Cl. A61M 5/31

(52) U.S. Cl. CPC A61M 5/3129

(57) ABSTRACT

The present disclosure is directed to pre-filled injection devices and related therapeutic formulations and methods that lack fatty acid particles. The therapeutic formulation may remain fatty acid particle free or substantially fatty acid particle free over time or when subjected to accelerated aging conditions.

### CROSS-REFERENCE TO RELATED APPLICATION

This application is a national phase application of PCT Application No. PCT/US2021/072413, internationally filed on Nov. 15, 2021, which claims the benefit of U.S. Provisional Application No. 63/114,192, filed on Nov. 16, 2020, which are herein incorporated by reference in their entireties for all purposes.

### FIELD

The present disclosure relates generally to pre-filled injection devices, and in particular, to pre-filled injection devices and related therapeutic formulations and methods that lack fatty acid particles.

### BACKGROUND

Pre-filled injection devices function to both store and deliver therapeutic formulations including drugs and/or biologics. Pre-filled injection devices generally offer cost savings to the pharmaceutical industry and may improve the safety, convenience, and efficacy of drug delivery. Biopharmaceuticals are an important class of pharmaceuticals that may increase the use of pre-filled injection devices, including syringes and auto injectors. As more pharmaceuticals and particularly biopharmaceuticals are utilized for delivery in pre-filled injection devices, the use of conventional pre-filled technology presents several challenges.

One challenge is the use of silicone (e.g., silicone oil) and/or other liquid lubricants. Conventionally, silicone provides a liquid seal between the stopper and the barrel. While silicone has traditionally been used to ensure that the force required to actuate a pre-filled injection device is minimized, the use of silicone as a lubricant poses a contamination risk. For example, silicone may contaminate the drug or biologic within the injection device. Additionally, the silicone may be injected into a patient along with the drug. Silicone may be of particular concern with biopharmaceuticals because it can cause aggregation of certain proteins, thereby rendering the biopharmaceutical unusable for injection.

Another challenge is the use of polysorbate and/or other surfactants that contain fatty acid esters. Conventionally, surfactants reduce the effect of protein adsorption to the silicone oil and/or reduce interfacial tension. However, such surfactants may generate undesirable particles, including fatty acid particles. This process may be catalyzed in biopharmaceutical formulations having lipase activity. In practice, these fatty acid particles may be indistinguishable from bacterial contamination, thus rendering the therapeutic formulation unusable.

Therefore, a need exists for formulations, methods, and pre-filled injection devices that lack fatty acid particles.

### SUMMARY

The present disclosure is directed to pre-filled injection devices and related therapeutic formulations and methods that lack fatty acid particles. The therapeutic formulation may remain fatty acid particle free or substantially fatty acid particle free over time or when subjected to accelerated aging conditions.

According to one example (“Example 1”), a pre-filled injection device is provided including a stopper, a barrel, a solid lubricant on at least one of the stopper and the barrel, the pre-filled injection device being free or substantially free of a liquid lubricant, and a therapeutic formulation having lipase activity and comprising at least about 1 mg/ml of one or more active pharmacological agents, wherein the therapeutic formulation is free or substantially free of fatty acid particles.

According to another example (“Example 2”), a method of reducing fatty acid particles is provided, the method including incorporating a therapeutic formulation having lipase activity and comprising at least about 1 mg/ml of one or more active pharmacological agents into a pre-filled injection device including a stopper, a barrel, and a solid lubricant on at least one of the stopper and the barrel, the pre-filled injection device being free or substantially free of a liquid lubricant, wherein the therapeutic formulation is free or substantially free of a surfactant.

According to yet another example (“Example 3”), a method of reducing the injection of fatty acid particles into a subject is provided, the method including administrating to a subject a therapeutically effective amount of a therapeutic formulation from a pre-filled injection device including a stopper, a barrel, and a solid lubricant on at least one of the stopper and the barrel, the pre-filled injection device being free or substantially free of a liquid lubricant, wherein the therapeutic formulation is free or substantially free of a surfactant, and the therapeutic formulation has lipase activity and comprises at least about 1 mg/ml of one or more active pharmacological agents.

According to yet another example (“Example 4”), a parenteral formulation is provided including at least about 1 mg/ml of one or more active pharmacological agents, wherein the formulation has lipase activity and is free or substantially free of fatty acid particles when stored at 2° C. to 8° C. for at least about 1 year.

According to yet another example (“Example 5”), a pre-filled injection device is provided including a stopper, a barrel, a solid lubricant on at least one of the stopper and the barrel, the pre-filled injection device being free or substantially free of a liquid lubricant, and a therapeutic formulation having lipase activity and comprising at least about 1 mg/ml of one or more active pharmacological agents, wherein the at least one therapeutic formulation is free or substantially free of a surfactant.

The foregoing Examples are just that and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The present invention will be better understood in view of the following non-limiting figures, in which:

FIG. 1 is an elevational view of an exemplary pre-filled syringe including a barrel, a plunger with a stopper, a needle, and a therapeutic formulation;

FIG. 2 is a partial cutaway view of the stopper of FIG. 1 having an elastomeric body at least partially covered by a solid lubricant;

FIG. 3 is a partial cutaway view of another stopper having an elastomeric body, an intermediate porous layer, and a solid lubricant; and

FIG. 4 is a schematic view of the therapeutic formulation of FIG. 1.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### Description of Various Embodiments

Persons skilled in the art will readily appreciate that various aspects of the present disclosure can be realized by any number of methods and apparatus configured to perform the intended functions. It should also be noted that the accompanying drawing figures referred to herein are not necessarily drawn to scale and may be exaggerated to illustrate various aspects of the present disclosure, and in that regard, the figures should not be construed as limiting.

### I. Pre-Filled Syringe

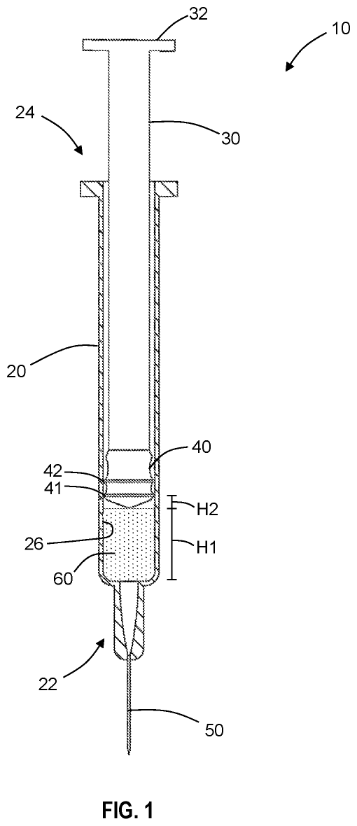

FIG. 1 depicts a drug injection device (e.g., syringe) 10 that may be pre-filled for storing and delivering at least one therapeutic formulation 60 to a patient. The illustrative syringe 10 includes a barrel 20, a plunger 30 having a stopper 40, and a piercing element (e.g., needle) 50, each of which is described further below. It is also within the scope of the present disclosure for the syringe 10 to be a “needleless” device having a Luer system (not shown). Other suitable drug injection devices within the scope of the present disclosure include auto-injectors and injectable pens, for example.

The barrel 20 of the syringe 10 contains the therapeutic formulation 60 and includes a distal end 22 that faces toward the patient, a proximal end 24 that faces away from the patient, and an inner surface 26 that faces inward toward the liquid therapeutic formulation 60. The barrel 20 may be formed of a hard material, such as a glass material (e.g., borosilicate glass), a ceramic material, one or more polymeric materials (e.g., polypropylene, polyethylene, and copolymers thereof), a metallic material, a plastic material (e.g., cyclic olefin polymers and cyclic olefin copolymers), and combinations thereof. In some embodiments, the barrel 20 has already been pre-filled with the therapeutic formulation 60 upon delivery to the user. The barrel 20 may contain about 0.5 mL to about 20 mL of the therapeutic formulation 60, but the syringe 10 may also be appropriately scaled to smaller doses or larger, multi-doses.

The plunger 30 of the syringe 10 is reciprocally movable within the barrel 20 to charge or discharge the therapeutic formulation 60 by moving the stopper 40. The plunger 30 includes a head 32 that extends from the proximal end 24 of the barrel 20. The stopper 40 is coupled to the opposing end of the plunger 30 near the distal end 22 of the barrel 20 and the needle 50. The illustrative stopper 40 of FIG. 1 contacts the inner surface 26 of the barrel 20 via one or more sealing ribs 41, 42, although any number of sealing ribs and/or non-sealing ribs may be present on the stopper 40.

As shown in FIG. 1, the stopper 40 may be positioned at a predetermined location in the barrel 20 relative to the therapeutic formulation 60. The therapeutic formulation 60 has a liquid height H1, which depends on the volume of the therapeutic formulation 60 in the barrel 20. The stopper 40 may be located at a predetermined stopper height or “headspace” H2 above the therapeutic formulation 60, which may be measured from the top surface of the therapeutic formulation 60 to the nearest sealing rib 41 of the stopper 40. The headspace H2 may be selected to control the amount of air in the barrel 20 between the stopper 40 and the therapeutic formulation 60. In some embodiments, the headspace H2 is less than about 25 mm, less than about 23 mm, less than about 21 mm, less than about 19 mm, less than about 17 mm, less than about 15 mm, less than about 13 mm, less than about 10 mm, less than about 8 mm, less than about 5 mm, less than about 3 mm, less than about 2 mm, less than about 1 mm, or less than about 0.5 mm. The headspace volume may be calculated by multiplying the headspace height H2 by the interior cross-sectional area of the barrel 20, less any volume of the stopper 40 that extends past the sealing rib 41 of the stopper 40 toward the therapeutic formulation 60. It may be advantageous to minimize the headspace H2 to reduce or avoid aggregation of the therapeutic formulation 60 in the syringe 10.

The stopper 40 should have low air and liquid permeability to minimize liquid leakage within the barrel 20 and the introduction of air between the stopper 40 and the inner surface 26 of the barrel 20 when charging or discharging the therapeutic formulation 60. In this way, the stopper 40 may resist bacterial contamination in the barrel 20. The stopper 40 should also possess low-friction slidability relative to the barrel 20 to facilitate the charging and discharging of the therapeutic formulation 60 inside the barrel 20. In some embodiments, the slide force between the stopper 40 and the barrel 20 may be less than 15 N, less than 10 N, or less than 5 N. The stopper 40 is described further in Section II below.

The needle 50 of the syringe 10 is coupled to the distal end 22 of the barrel 20. The needle 50 is configured to pierce the patient's skin and inject the therapeutic formulation 60 into the patient by pressing the plunger 30. In the event that the needle 50 is removable, care should be taken to minimize any bacterial contamination in the barrel 20 when coupling the needle 50 to the barrel 20 and/or when uncoupling the needle 50 from the barrel 20.

The interior of the syringe 10 (not including the needle 50, as explained below) is free of liquid lubricants (i.e., “lubricant free”) or substantially free of liquid lubricants (i.e., “substantially lubricant free”). In particular, the barrel 20 and the stopper 40 of the syringe 10 are free or substantially free of silicone (e.g., silicone oil, silicone grease). As used herein, the phrases “lubricant free” and “free of liquid lubricants” mean that the barrel 20 and the stopper 40 contain no liquid lubricant of any kind, either intentionally or accidentally (i.e., 0 picograms (pg) of lubricants), or contain only a trace amount of liquid lubricant that is undetectable by any known measuring equipment or method. The phrases “substantially lubricant free” and substantially free of liquid lubricants” mean that the barrel 20 and the stopper 40 contain an insignificant but measurable amount of liquid lubricants, such as about 5 μg or less, about 4 μg or less, about 3 μg or less, about 2 μg or less, or about 1 μg or less. In certain embodiments, liquid lubricants are present on the barrel 20 and/or the stopper 40 from 0 μg to about 5 μg, from about 1 μg to about 5 μg, from about 2 μg to about 5 μg, from about 3 μg to about 5 μg, or from about 4 μg to about 5 μg. The absence or substantial absence of liquid lubricants can be measured using gas chromatography (GC) mass spectrometry, inductively coupled plasma (ICP) mass spectrometry, and/or by the amount of particles in the barrel 20 that are measured in water for injection (WFI) after the WFI has been exposed to a fully assembled syringe (e.g., a glass barrel 20 and stopper 40 and alternatively at least one therapeutic compound). In some embodiments, the amount of particles in the barrel 20 may be less than about 600 particles/ml for particles greater than 10 pm in size or less than 60 particles/ml for particles greater than 25 pm in size when measured in WFI. The needle 50 of the syringe 10 may have a lubricant to ease insertion into the patient's skin without impacting the ability for the rest of the syringe 10 to be free “lubricant free” or “substantially lubricant free”, as described above.

### II. Syringe Stopper

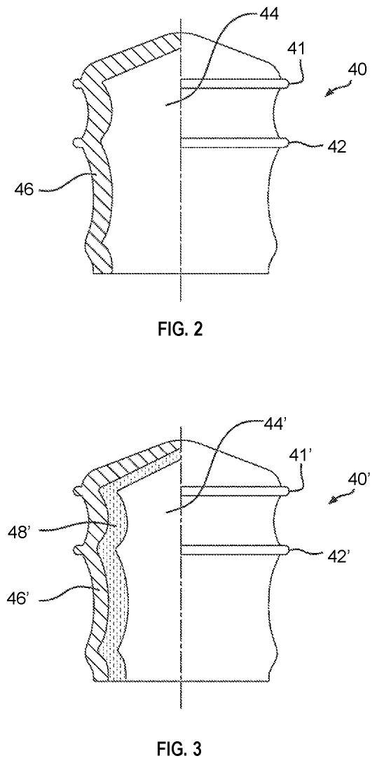

Referring next to FIG. 2, the stopper 40 is shown in more detail and includes an elastomeric body 44 at least partially covered by a solid lubricant 46. The solid lubricant 46 may be designed to provide a low coefficient of friction with the barrel 20 (FIG. 1), compliance, low extractables and leachables (in particular, low metal-ion extractables and leachables), and/or good barrier properties against any extractables and leachables in the elastomeric body 44.

The elastomeric body 44 of the stopper 40 may comprise any suitable elastomer, such as butyl rubber, bromobutyl rubber, chlorobutyl rubber, silicone, nitrile, styrene butadiene, polychloroprene, ethylene propylene diene, fluoroelastomers and combinations thereof. In other embodiments, the stopper 40 may be constructed of non-elastomeric materials, such as plastics (e.g., polypropylene, polycarbonate, and polyethylene), thermoplastics, and fluoropolymer materials such as ethylene-(perfluoro-ethylene-propene) copolymer (EFEP), polyvinylidene difluoride (PVDF), and perfluoroalkoxy polymer resin (PFA).

The solid lubricant 46 of the stopper 40 may comprise a low coefficient of friction polymer layer, which may have a coefficient of friction of about 0.08 to about 0.8 against the glass of the barrel 20. The solid lubricant 46 may be constructed of a fluoropolymer including, but not limited to, polytetrafluoroethylene (PTFE), expanded polytetrafluoroethylene (ePTFE), densified ePTFE, and copolymers and combinations thereof. Other materials for use as the solid lubricant 46 include, but are not limited to, fluorinated ethylene propylene (FEP), ethylene tetrafluoroethylene (ETFE), polyvinylfluoride, polyvinylidene fluoride (e.g., poly(vinylidene fluoride-co-tetrafluoroethylene) (VDF-co-TFE), poly(vinylidene fluoride-co-trifluoroethylene) (VDE-co-TrFE)), perfluoropropylvinylether, perfluoroalkoxy polymers, polyethylene (e.g., expanded ultra-high molecular weight polyethylene (eUHMWPE)), polypropylene, poly (p-xylylene) (PPX), polylactic acid (PLA), poly(L-lactic acid) (PLLA), poly(D-lactic acid) (PDLA), and copolymers and combinations thereof, which may be expanded if desired. It is to be appreciated that, the solid lubricant 46 as described herein may be on, cover, or at least partially cover at least one of the stopper 40 and the barrel 20.

The stopper 40 of FIG. 2 may be manufactured by thermoforming the solid lubricant 46 from a densified ePTFE film and then molding (e.g., injection molding, compression molding) the elastomeric body 44 onto the thermoformed solid lubricant 46. In another embodiment, the stopper 40 of FIG. 2 may be manufactured by a direct molding process, in which a sheet of the solid lubricant 46 is placed in a heated mold together with the material for elastomeric body 44 to simultaneously vulcanize the elastomeric body 44, if applicable, and form the stopper 40. It is also within the scope of the present disclosure to pre-treat or post-treat the solid lubricant 46 with chemical etching, plasma treating, corona treatment, roughening, or the like to improve the bonding of the solid lubricant 46 to the elastomeric body 44.

Referring next to FIG. 3, another stopper 40′ is shown, with like reference numerals identifying like elements. The stopper 40′ includes an elastomeric body 44′ and a solid lubricant 46′ (also referred to as a barrier layer), as well as an intermediate porous layer 48′. The porous layer 48′ may comprise or be formed of ePTFE or other porous expanded and advantageously fibrillizing fluoropolymers. The adjacent elastomeric body 44′ and/or solid lubricant 46′ may at least partially penetrate the intermediate porous layer 48′, and the degree of penetration may be controlled to achieve desired strength, toughness, compliance and stability for the desired application.

The stopper 40′ of FIG. 3 may be manufactured by forming the porous layer 48′, coating, laminating, imbibing, or otherwise applying the solid lubricant 46′ onto and/or into the porous layer 48′ to create a multi-layered or composite film, and then molding (e.g., injection molding, compression molding) the elastomeric body 44′ onto the film such that the elastomeric body 44′ at least partially penetrates the pores of the porous layer 48′. It is also within the scope of the present disclosure to pre-treat or post-treat the solid lubricant 46′ and/or the porous layer 48′ with chemical etching, plasma treating, corona treatment, roughening, or the like to improve the bonding of the solid lubricant 46′ to the elastomeric body 44′.

### III. Therapeutic Formulation

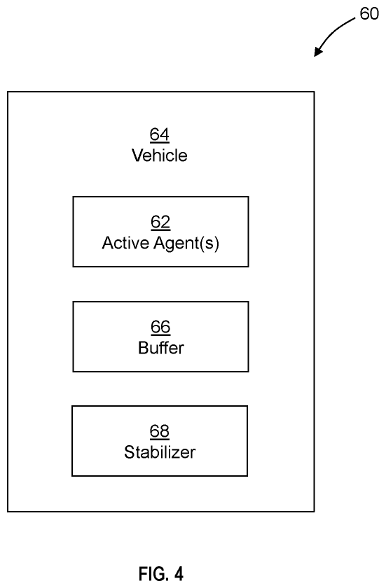

The therapeutic formulation 60 is shown schematically in FIG. 4. The therapeutic formulation 60 has “lipase activity”, meaning that the therapeutic formulation 60 contains one or more lipase enzymes capable of hydrolyzing fatty acid esters into free fatty acids. Lipase activity may be measured through fatty acid titration or other suitable techniques. One suitable lipase activity assay involves hydrolyzing a 4-methylumbelliferyl oleate (4MuO) lipase substrate to yield a fluorescent 4-methylumbelliferone (4Mu) product and detecting the 4Mu product by fluorescence emission (Jahn et al., “Measuring Lipolytic Activity to Support Process Improvements to Manage Lipase-Mediated Polysorbate Degradation”, Pharm Res. 2020; 37(6):118). Other suitable lipase activity assays involve complexing and detecting liberated fatty acids with Rhodamine B, or reacting the lipase substrate 4-nitrophenyl palm itate or 4-nitrophenyl butyrate and detecting the released chromogenic reaction product 4-nitrophenol (4 Np).

The therapeutic formulation 60 of FIG. 4 includes one or more active pharmacological agents 62, more specifically active biopharmaceuticals agents. The active agents 62 may be used for use in the treatment of inflammatory diseases including, but not limited to, inrheumatoid arthritis (RA), psoriasis, inflammatory bowel disease (IBD), and ocular inflammatory disease. Active agents 62 include, but are not limited to, proteins, antibodies, cytokines, growth factors, coagulation factors, proteases, kinases, phosphatases, vaccines, peptides, small interfering RNAs (siRNAs), small interfering DNAs (siDNAs), messenger RNAs (mRNAs), aptamers, and/or a combination thereof. The active agent(s) 62 may be present in the therapeutic formulation 60 at a concentration of at least about 1 mg/ml, such as a concentration from about 1 mg/ml to about 200 mg/ml, from about 10 mg/ml to about 200 mg/ml, from about 20 mg/ml to about 200 mg/ml, from about 40 mg/ml to about 200 mg/ml, from about 60 mg/ml to about 200 mg/ml, from about 80 mg/ml to about 200 mg/ml, from about 100 mg/ml to about 200 mg/ml, from about 120 mg/ml to about 200 mg/ml, and/or from about 150 mg/ml to about 200 mg/ml. Specific active agents 62 are set forth in Section IV below.

The therapeutic formulation 60 of FIG. 4 also includes a vehicle 64 (e.g., solvent, diluent) capable of conveying the active agent 62 to the patient during injection. Suitable solvents include, for example, water, acetic acid, propylene glycol, ethylene glycol, polyethylene glycol, benzyl benzoate, and combinations thereof.

The therapeutic formulation 60 may also include one or more excipients. The excipients may be configured to protect, support, or enhance processability, stability, sterility, bioavailability, product identification, effectiveness, delivery, and/or storage integrity.

One excipient is a buffer 66 including, for example, phosphate (e.g., phosphate buffered saline (PBS), acetate, histidine, and tris. The buffer 66 may have a pH from about 4.0 to about 9.5, from about 4.5 to about 9.0, from about 5.0 to about 8.5, from about 5.5 to about 8.0, from about 5.5 to about 7.5, from about 5.5 to about 7.0, and/or from about 5.5 to about 6.5.

Another excipient is a stabilizer 68 including, for example, sugars (e.g., sucrose, trehalose, maltose, and lactose), polyols (e.g., mannitol, sorbitol, and glycerol), and amino acid salts (e.g., histidine, arginine, and glycine). The concentration of sugar in the therapeutic formulation 60 may be from 0 wt. % to about 15 wt. %, from about 0.1 wt. % to about 15 wt. %, from about 1 wt. % to about 15 wt. %, from about 1.5 wt. % to about 10 wt. %, from about 2 wt. % to about 10 wt. %, from about 3 wt. % to about 10 wt. %, and/or from about 5 wt. % to about 10 wt. %. The concentration of polyol in the therapeutic formulation 60 may be from 0 wt. % to about 5 wt. %, from about 0.1 wt. % to about 5 wt. %, from about 1 wt. % to about 5 wt. %, from about 1.5 wt. % to about 5 wt. %, from about 2 wt. % to about 5 wt. %, and/or from about 3 wt. % to about 5 wt. %. The concentration of amino acid salts in the therapeutic formulation 60 may be from 0 wt. % to about 5 wt. %, from about 0.1 wt. % to about 5 wt. %, from about 1 wt. % to about 5 wt. %, from about 1.5 wt. % to about 5 wt. %, from about 2 wt. % to about 5 wt. %, and/or from about 3 wt. % to about 5 wt. %.

The therapeutic formulation 60 of FIG. 4 is free of surfactants (i.e., “surfactant free”) or substantially free of surfactants (i.e., “substantially surfactant free”). In particular, the therapeutic formulation 60 is free or substantially free of polysorbate surfactants, including polysorbate 20, polysorbate 40, polysorbate 60, polysorbate 80, and combinations thereof. Such surfactants are generally used to lower surface tension and/or interfacial tension and prevent therapeutics from adsorbing onto surfaces and/or interfaces. As used herein, the phrases “surfactant free” and “free of surfactants” mean that the therapeutic formulation 60 contains no surfactants of any kind, either intentionally or accidentally (i.e., 0 wt. % surfactants), or contains only a trace amount of surfactants that is undetectable by any known measuring equipment or method. The phrases “substantially surfactant free” and “substantially free of surfactants” mean that the therapeutic formulation 60 contains an insignificant but measurable amount of surfactants, such as about 0.1 wt. % or less, about 0.075 wt. % or less, about 0.05 wt. % or less, about 0.025 wt. % or less, about 0.01 wt. % or less, about 0.005 wt. % or less, or about 0.001 wt. % or less. In certain embodiments, the concentration of surfactants in the therapeutic formulation 60 may be from 0 wt. % to about 0.1 wt. %, from 0 wt. % to about 0.075 wt. %, from 0 wt. % to about 0.05 wt. %, from 0 wt. % to about 0.025 wt. %, from 0 wt. % to about 0.01 wt. %, from 0 wt. % to about 0.005 wt. %, and/or from 0 wt. % to about 0.001 wt. %.

The therapeutic formulation 60 of FIG. 4 is also free of fatty acid particles (i.e., “fatty acid particle free”) or substantially free of fatty acid particles (i.e., “substantially fatty acid particle free”). As used herein, the terms “fatty acid particle free” and “free of fatty acid particles” mean that the therapeutic formulation 60 contains no fatty acid particles of any kind, either intentionally or accidentally (i.e., 0 particles), or contains only fatty acid particles too small to be detected by any known measuring equipment or method. The terms “substantially fatty acid particle free” and “substantially free of fatty acid particles” mean that the therapeutic formulation 60 contains an insignificant but measurable number of fatty acid particles. For fatty acid particles of a size greater than 25 microns, the therapeutic formulation 60 may contain about 600 particles or less, about 300 particles or less, about 100 particles or less, about 20 particles or less, about 5 particles or less, about 2 particles or less, or about 1 particle. For fatty acid particles of a size greater than 10 microns, the therapeutic formulation 60 may contain about 6000 particles or less, about 3000 particles or less, about 1000 particles or less, about 200 particles or less, about 50 particles or less, about 20 particles or less, or about 10 particles or less. Lower fatty acid particle counts may be required for ocular applications, whereas higher fatty acid particle counts may be suitable for other applications. These fatty acid particle counts may be obtained through visual inspection in a light box, such as the Seidenader V90-T, micro-flow imaging (MFI), or another suitable technique.

The therapeutic formulation 60 may remain fatty acid particle free or substantially fatty acid particle free over time. For example, the therapeutic formulation 60 may remain fatty acid particle free or substantially fatty acid particle free when stored at 2° C. to 8° C. for about 1 year, about 2 years, about 3 years, or longer.

The therapeutic formulation 60 may also remain fatty acid particle free or substantially fatty acid particle free when subjected to higher temperatures or accelerated aging conditions. For example, the therapeutic formulation 60 may remain fatty acid particle free or substantially fatty acid particle free even when heated to 40° C. for 2 months or longer and then cooled to 5° C. for at least 24 hours.

### IV. Active Agents

As noted above, the therapeutic formulation 60 includes one or more active agents 62, which may include biomolecules such as proteins, antibodies, cytokines, growth factors, coagulation factors, proteases, kinases, phosphatases, vaccines, peptides, small interfering RNAs (siRNAs), small interfering DNAs (siDNAs), messenger RNAs (mRNAs), aptamers, and/or any combination thereof. For instance, the therapeutic formulation 60 may include one or more of the following active agents 62:

Examples of antibodies, antisense, RNA interference, or gene therapy made to protein targets or gene(s) of: Ataxia Telangiectasia Mutated, Tumor Protein p53, Checkpoint kinase 2, breast cancer susceptibility protein, Double-strand break repair protein, DNA repair protein RAD50, Nibrin, p53-binding protein, Mediator of DNA damage checkpoint protein, H2A histone family member X, Microcephalin, C-terminal-binding protein 1, Structural maintenance of chromosomes protein 1A; Esterases; Phosphatases; Examples of Ion channels include but are not limited to: ligand-gated ion channels, voltage-gated ion channels; Examples of growth factors include but are not limited to: nerve growth factor (NGF), vascular endothelial growth factor (VEGF), platelet-derived growth factor (PDGF), C-fos-induced growth factor (FIGF), platelet activating factor (PAF), transforming growth factor beta (TGF-b), b, one morphogenetic proteins (BMPs), Activin, inhibin, fibroblast growth factors (FGFs), granulocyte-colony stimulating factor (G-CSF), granulocyte-macrophage colony stimulating factor (GM-CSF), glial cell line-derived neurotrophic factor (GDNF), growth differentiation factor-9 (GDF9), epidermal growth factor (EGF), transforming growth factor-a (TGF-a), growth factor (KGF), migration-stimulating factor (MSF), hepatocyte growth factor-like protein (FIGFLP), hepatocyte growth factor (FIGF), hepatoma-derived growth factor (FIDGF), Insulin-like growth factors; Examples of G Protein-Coupled Receptors (GPCR) include but are not limited to: Adenosine receptor family, Adrenergic receptor family, Angiotensin II receptor, Apelin receptor, Vasopressin receptor family, Brain-specific angiogenesis inhibitor family, Bradykinin receptor family, Bombesin receptor family, Complement component 3a receptor 1, Complement component 5a receptor 1, Calcitonin receptor family, Calcitonin receptor-like family, Calcium-sensing receptor, Cholecystokinin A receptor (CCK1), Cholecystokinin B receptor (CCK2), Chemokine (C-C motif) receptor family, Sphingosine 1-phosphate receptor family, Succinic receptor, Cholinergic receptor family. Chemokine-like receptor family, Cannabinoid receptor family, Corticotropin releasing hormone receptor family, prostaglandin D2 receptor, Chemokine C-X3-C receptor family, Chemokine (C-X-C motif) receptor family, Burkitt lymphoma receptor, Chemokine (C-X-C motif) receptor family, Cysteinyl leukotriene receptor 2 (CYSLT2), chemokine receptor (FY), Dopamine receptor family, G protein-coupled receptor 183 (GPR183), Lysophosphatidic acid receptor family, Endothelin receptor family, Coagulation factor II (thrombin) receptor family, Free fatty acid receptor family, Formylpeptide receptor family, Follicle stimulating hormone receptor (FSFIR), gamma-aminobutyric acid (GABA) B receptor, Galanin receptor family, Glucagon receptor, Growth hormone releasing hormone receptor (GFHRFH), Ghrelin receptor (ghrelin), Growth hormone secretagogue receptor 1 b (GHSRI b), Gastric inhibitory polypeptide receptor (GIP), Glucagon-like peptide receptor family, Gonadotropin releasing hormone receptor (GnRH), pyroglutamylated RFamide peptide receptor (QRFPR), G protein-coupled bile acid receptor 1 (GPBA), Flydroxycarboxylic acid receptor family, Lysophosphatidic acid receptor 4 (LPA4) Lysophosphatidic acid receptor 5 (GPR92), G protein-coupled receptor 79 pseudogene (GPR79), Flydroxycarboxylic acid receptor 1 (FHCA1), G-protein coupled receptor (C5L2, FFA4, FFA4, FFA4, GPER, GPR1, GPR101, GPR107, GPR119, GPR12, GPR123, GPR132, GPR135, GPR139, GPR141, GPR142, GPR143, GPR146, GPR148, GPR149, GPR15, GPR150, GPR151, GPR152, GPR157, GPR161, GPR162, GPR17, GPR171, GPR173, GPR176, GPR18, GPR182, GPR20, GPR22, GPR25, GPR26, GPR27, GPR3, GPR31, GPR32, GPR35, GPR37L1, GPR39, GPR4, GPR45, GPR50, GPR52, GPR55, GPR6, GPR61, GPR65, GPR75, GPR78, GPR83, GPR84, GPR85, GPR88, GPR97, TM7SF1), Metabotropic glutamate receptor family, Gastrin releasing peptide receptor (BB2), Orexin receptor family, Flistamine receptor family, 5-hydroxytryptamine receptor family, KISS1-derived peptide receptor (kisspeptin), Leucine-rich repeat-containing G protein-coupled receptor family, horiogonadotropin receptor (LFH), Leukotriene B4 receptor (BLT1), Adenylate Cyclase Activating Polypeptide 1 Receptor 1 (mPAC1), Motilin receptor, Melanocortin receptor family, Melanin concentrating hormone receptor 1 (MCH1), Neuropeptide Y1 receptor (Y1), Neuropeptide Y2 receptor (NPY2R), Opioid receptor family, Oxytocin recepter (OT), P2Y Purinoceptor 12 (mP2Y12), P2Y Purinoceptor 6 (P2Y6), Pancreatic polypeptide receptor family, Platelet-activating factor receptor family, Prostaglandin E receptor family, Prostanoid IP1 receptor (IP 1), MAS-related GPR, member family, Rhodopsin (Rhodopsin), Relaxin family peptide receptor family, Somatostatin receptor family, Tachykinin receptor family, Melatonin receptor family, Urotensin receptor family, Vasoactive intestinal peptide receptor 1 (mVPACI), Neuromedin B Receptor (BB1), Neuromedin U receptor 1 (NMU1), Neuropeptides B/W receptor family, Neuropeptide FF receptor 1 (NPFF1), neuropeptide S receptor 1 (NPS receptor), Neuropeptide Y receptor family, Neurotensin receptor 1 (NTS1), Opsin 5 (OPN5), Opioid receptor-like receptor (NOP), Oxoeicosanoid (OXE) receptor 1 (OXE), Oxoglutarate (alpha-ketoglutarate) receptor 1 (OXGR1), Purinergic receptor family, Pyrimidinergic receptor family, Prolactin releasing hormone receptor (PRRP), Prokineticin receptor family, Platelet activating receptor (PAF), Prostaglandin F receptor family, Prostaglandin 12 (prostacyclin) receptor family, Parathyroid hormone receptor family, muscarinic 4 (rM4), Prostanoid DP2 receptor (rGPR44), Prokineticin receptor family, Relaxin family peptide receptor family, Secretin receptor (secretin), Smoothened, Frizzled class receptor (Smoothened), trace amine associated receptor family, Tachykinin family, Thromboxane A2 receptor (TP), Thyrotropin-releasing hormone receptor (TRH 1), Thyroid Stimulating Flormone Receptor (TSFI); Examples of Protein kinases include but are not limited to: AP2 associated kinase, Flomo sapiens ABL proto oncogene 1-non-receptor tyrosine-protein kinase family, c-abl oncogene 1 receptor tyrosine kinase family, v-abl Abelson murine leukemia viral oncogene homolog 2, activin A receptor family, chaperone-ABC1 activity of bd complex homolog (S. pombe) (ADCK3), aarF domain containing kinase 4 (ADCK4), v-akt murine thymoma viral oncogene homolog family, anaplastic lymphoma receptor tyrosine kinase family, protein kinase A family, protein kinase B family, ankyrin repeat and kinase domain containing 1 (ANKK1), NUAK family-SNF1-like kinase, mitogen-activated protein kinase family aurora kinase A (AURKA), aurora kinase B (AURKB), aurora kinase C (AURKC), AXL receptor tyrosine kinase (AXL), BMP2 inducible kinase (BIKE), B lymphoid tyrosine kinase (BLK), bone morphogenetic protein receptor family, BMX non-receptor tyrosine kinase (BMX), v-raf murine sarcoma viral oncogene homolog B1 (BRAF), protein tyrosine kinase 6 (BRK), BR serine/threonine kinase family, Bruton agammaglobulinemia tyrosine kinase (BTK), calcium/calmodulin-dependent protein kinase family, cyclin-dependent kinase family, cyclin-dependent kinase-like family, CHK1 checkpoint homolog (S. pombe) (CHEK1), CHK2 checkpoint homolog (S. pombe) (CHEK2), Insulin receptor, isoform A (INSR), Insulin receptor, isoform B (INSR), rho-interacting serine/threonine kinase (CIT), v-kit Hardy-Zuckerman 4 feline sarcoma viral oncogene homolog (KIT), CDC-Like Kinase family-Hepatocyte growth factor receptor (MET), Proto-oncogene tyrosine-protein kinase receptor, colony-stimulating factor family receptor, c-src tyrosine kinase (CSK), casein kinase family, megakaryocyte-associated tyrosine kinase (CTK), death-associated protein kinase family, doublecortin-like kinase family, discoidin domain receptor tyrosine kinase, dystrophia myotonica-protein kinase (DMPK), dual-specificity tyrosine-(Y)-phosphorylation regulated kinase family, epidermal growth factor receptor family, eukaryotic translation initiation factor 2-alpha kinase 1 (EIF2AK1), EPH receptor family, Ephrin type-A receptor family, Ephrin type-B receptor family, v-erb-b2 erythroblastic leukemia viral oncogene homolog family, mitogen-activated protein kinase family, endoplasmic reticulum to nucleus signaling 1 (ERN1), PTK2 protein tyrosine kinase 2 (FAK), fer (fps/fes related) tyrosine kinase (FER). feline sarcoma oncogene (FES), Fibroblast growth factor receptor family, Gardner-Rasheed feline sarcoma viral (v-fgr) oncogene homolog (FGR), fms-related tyrosine kinase family, Fms-related tyrosine kinase family, fyn-related kinase (FRK), FYN oncogene related to SRC, cyclin G associated kinase (GAK), eukaryotic translation initiation factor 2 alpha kinase, Growth hormone receptor. G protein-coupled receptor kinase 1 (GRK1), G protein-coupled receptor kinase family, glycogen synthase kinase family, germ cell associated 2 (haspin) (FIASPIN), Flemopoietic cell kinase (FICK), homeodomain interacting protein kinase family, mitogen-activated protein kinase family, hormonally up-regulated Neu-associated kinase (FIUNK), intestinal cell (MAK-like) kinase (ICK), Insulin-like growth factor 1 receptor (IGF1 R), conserved helix-loop-helix ubiquitous kinase (IKK-alpha), inhibitor of kappa light polypeptide gene enhancer in B-cells-kinase beta family, insulin receptor (I NSR), insulin receptor-related receptor (INS RR), interleukin-1 receptor-associated kinase family, IL2-inducible T-cell kinase (ITK), Janus kinase family, Kinase Insert Domain Receptor, v-kit Flardy-Zuckerman 4 feline sarcoma viral oncogene homolog, lymphocyte-specific protein tyrosine kinase (LCK), LIM domain kinase family, serine/threonine kinase family leucine-rich repeat kinase family, v-yes-1 Yamaguchi sarcoma viral related oncogene homolog (LYN), male germ cell-associated kinase (MAK), MAP/microtubule affinity-regulating kinase family, microtubule associated serine/threonine kinase family, maternal embryonic leucine zipper kinase, c-mer proto-oncogene tyrosine kinase (MERTK), met proto-oncogene (hepatocyte growth factor receptor), MAP kinase interacting serine/threonine kinase family, myosin light chain kinase family, mixed lineage kinase domain-like protein isoform, CDC42 binding protein kinase family, serine/threonine kinase family, macrophage stimulating 1 receptor (c-met-related tyrosine kinase) (MST1 R), mechanistic target of rapamycin (serine/threonine kinase) (MTOR), muscle-skeletal-receptor tyrosine kinase (MUSK), myosin light chain kinase family, NIMA (never in mitosis gene a)-related kinase family, serine/threonine-protein kinase NIM1 (NIM1), nemo-like kinase (NLK), oxidative-stress responsive 1 (OSR1), p21 protein (Cdc42/Rac)-activated kinase family, PAS domain containing serine/threonine kinase, Platelet-derived growth factor receptor family, 3-phosphoinositide dependent protein kinase-1 (PDPK1), Calcium-dependent protein kinase 1, phosphorylase kinase gamma family, Phosphatidylinositol 4,5-bisphosphate 3-kinase, phosphoinositide-3-kinase family, phosphatidylinositol 4-kinase family phosphoinositide kinase, FYVE finger containing, Pim-1 oncogene (PIM 1), pim-2 oncogene (PIM2), pim-3 oncogene (PIM3), phosphatidylinositol-4-phosphate 5-kinase family, phosphatidylinositol-5-phosphate 4-kinase family protein kinase, membrane associated tyrosine/threonine 1 (PKMYT1), protein kinase N family, polo-like kinase family, protein kinase C family, protein kinase D family, cGMP-dependent protein kinase family, eukaryotic translation initiation factor 2-alpha kinase 2 (PRKR), X-linked protein kinase (PRKX), Prolactin receptor (PRLR), PRP4 pre-mRNA processing factor 4 homolog B (yeast) (PRP4), PTK2B protein tyrosine kinase 2 beta (PTK2B), SIK family kinase 3 (QSK), v-raf-1 murine leukemia viral oncogene homolog 1 (RAF1), Neurotrophic tyrosine kinase receptor type family, receptor (TNFRSF)-interacting serine-threonine kinase family, dual serine/threonine and tyrosine protein kinase (RIPK5), Rho-associated, coiled-coil containing protein kinase family, c-ros oncogene 1, receptor tyrosine kinase (ROS1), ribosomal protein S6 kinase family, SFI3-binding domain kinase 1 (SBK1), serum/glucocorticoid regulated kinase family, Putative uncharacterized serine/threonine-protein kinase (Sugen kinase 1 10) (SgK110), salt-inducible kinase family, SNF related kinase (SNRK), src-related kinase, SFRS protein kinase family, Spleen tyrosine kinase (SYK), TAO kinase family, TANK-binding kinase 1 (TBK1), tec protein tyrosine kinase (TEC), testis-specific kinase 1 (TESK1), transforming growth factor, beta receptor family, tyrosine kinase with immunoglobulin like and EGF-like domains 1 (TIE1), TEK tyrosine kinase, endothelial (TIE2), Angiopoietin-1 receptor (Tie2), tousled-like kinase family, TRAF2 and NCK interacting kinase (TNIK), non-receptor tyrosine kinase family, TNNI3 interacting kinase (TNNI3K), transient receptor potential cation channel, testis-specific serine kinase family, TTK protein kinase (TTK), TXK tyrosine kinase (TXK), Tyrosine kinase 2 (TYK2), TYR03 protein tyrosine kinase (TYR03), unc-51-like kinase family, phosphatidylinositol 3-kinase, vaccinia related kinase 2 (VRK2), WEE1 homolog family, WNK lysine deficient protein kinase family, v-yes-1 Yamaguchi sarcoma viral oncogene homolog 1 (YES), sterile alpha motif and leucine zipper containing kinase AZK (ZAK), zeta-chain (TCR) associated protein kinase 70 kDa (ZAP70); Examples of nuclear hormone receptors include but are not limited to: Androgen receptor (AR), Estrogen related receptor alpha (ESRRA), Estrogen receptor 1 (ESR1), Nuclear receptor subfamily 1-group H-member 4 (NR1 H4), Nuclear receptor subfamily 3-group C-member 1 (glucocorticoid receptor) (NR3C1), Nuclear receptor subfamily 1-group H-member 3 (Liver X receptor a) (NR1 H3), Nuclear receptor subfamily 1-group H-member 2 (Liver X receptor b) (NR1 H2), Nuclear receptor subfamily 1-group H-member 2 (Liver X receptor b) (NR1 H2), Nuclear receptor subfamily 3-group C-member 2 (Mineralcorticoid receptor) (NR3C2), Peroxisome Proliferator Activated Receptor alpha (PPARA), Peroxisome Proliferator Activated Receptor gamma (PPARG), Peroxisome Proliferator Activated Receptor delta (PPARD), Progesterone receptor a (PGR), Progesterone receptor b (PGR), Retinoic acid receptor-alpha (RARA), Retinoic acid receptor-beta (RARB), Retinoid X receptor-alpha (RXRA), Retinoid X receptor-gamma (RXRG), Thyroid hormone receptor-alpha (THRA), Thyroid hormone receptor-beta (THRB), Retinoic acid-related orphan receptor, Liver X receptor, Farnesoid X receptor, Vitamin D receptor, Pregnane X receptor, Constitutive androstane receptor, Hepatocyte nuclear factor 4, Oestrogen receptor, Oestrogen-related receptor, Glucocortioic receptor, Nerve growth factor-induced-B, Germ cell nuclear factor; Examples of Epigenetic targets include but are not limited to: ATPase family AAA domain-containing protein 2 (ATAD2A), ATPase family-AAA domain containing 2B (ATAD2B), ATPase family AAA domain containing-2B (ATAD2B), bromodomain adjacent to zinc finger domain-1 A (BAZ1 A), bromodomain adjacent to zinc finger domain-1 B (BAZ1 B), bromodomain adjacent to zinc finger domain-2A (BAZ2A), bromodomain adjacent to zinc finger domain-2A (BAZ2A), bromodomain adjacent to zinc finger domain-2B (BAZ2B), bromodomain-containing protein 1 (BRD1), Bromodomain containing protein 2-1 st bromodomain (BRD2), Bromodomain containing protein 2-1st & 2nd bromodomains (BRD2), bromodomain-containing protein 2 isoform 1-bromodomain 2 (BRD2(2)), bromodomain-containing protein 3-bromodomain 1 (BRD3(1)), Bromodomain-containing protein 3-1 st bromodomain (BRD3), Bromodomain-containing protein 3-1 st & 2nd bromodomains (BRD3), bromodomain-containing protein 3-bromodomain 2 (BRD3(2)), Bromodomain containing protein 4-1st bromodomain (BRD4), bromodomain-containing protein 4 isoform long-bromodomains 1 and 2 (BRD4(1 -2)), bromodomain-containing protein 4 isoform long-bromodomain 2 (BRD4(2)), bromodomain-containing protein 4 isoform short (BRD4(full-length-short-iso.)), Bromodomain containing protein 7 (BRD7), bromodomain containing 8-bromodomain 1 (BRD8(1)), bromodomain containing 8-bromodomain 2 (BRD8(2)), bromodomain-containing protein 9 isoform 1 (BRD9), Bromodomain containing testis-specific-1 st bromodomain (BRDT), Bromodomain containing testis-specific-1 st & 2nd bromodomains (BRDT), bromodomain testis-specific protein isoform b-bromodomain 2 (BRDT(2)), bromodomain and PHD finger containing-1 (BRPF1), bromodomain and PHD finger containing-3 (BRPF3), bromodomain and PHD finger containing-3 (BRPF3), Bromodomain and WD repeat-containing 3-2nd bromodomain (BRWD3(2)), Cat eye syndrome critical region protein 2 (CECR2), CREB binding protein (CREBBP), E1A binding protein p300 (EP300), EP300 (EP300), nucleosome-remodeling factor subunit BPTF isoform 1 (FALZ), Nucleosome-remodeling factor subunit BPT (FALZ), Euchromatic histone-lysine N-methyltransferase 2 (EHMT2), Histone Acetyltransferase-KAT2A (GCN5L2), Euchromatic histone-lysine N-methyltransferase 1 (EHMT1), Histone-lysine N-methyltransferase MLL (MLL), Polybromo 1-1 st bromodomain (PB1 (1)), Polybromo 1-2nd bromodomain (PB1 (2)), polybromo 1-bromodomain 2 (PBRM1 (2)), polybromo 1-bromodomain 5 (PBRM1 (5)), Histone acetyltransferase KAT2B (PCAF), PH-interacting protein-1 st bromodomain (PHIP(1)), PH-interacting protein-2nd bromodomain (PHIP(2)), Protein kinase C-binding protein 1 (PRKCBP1), Protein arginine N-methyltransferase 3 (PRMT3), SWI/SNF related-matrix associated-actin dependent regulator of chromatin-subfamily a-member 2 (SMARCA2), SWI/SNF related-matrix associated-actin dependent regulator of chromatin-subfamily a-member 4 (SMARCA4), Nuclear body protein-SP110 (SP110), Nuclear body protein-SP140 (SP140), Transcription initiation factor TFIID subunit 1 (TAF1 (1-2)), TAF1 RNA polymerase II-TATA box binding protein (TBP)-associated factor-250kDa-bromodomain 2 (TAF1 (2)), Transcription initiation factor TFIID subunit 1-like-1 st bromodomain (TAF1 L(1)), Transcription initiation factor TFIID subunit 1-like-2nd bromodomain (TAF1 L(2)), tripartite motif containing 24 (TRIM24(Bromo.)), tripartite motif containing 24 (TRIM24(PFID-Bromo.)), E3 ubiquitin-protein ligase TRIM33 (TRIM33), tripartite motif containing 33 (TRIM33(PFID-Bromo.)), WD repeat 9-1 st bromodomain (WDR9(1)), WD repeat 9-2nd bromodomain (WDR9(2)); membrane transport proteins including but not limited to ATP-binding cassette (ABC) superfamily, solute carrier (SLC) superfamily, multidrug resistance protein 1 (P-glycoprotein), organic anion transporter 1, and protein such as EAAT3, EAAC1, EAAT1, GLUT1, GLUT2, GLUT9, GLUT10, rBAT, AE1, NBC1, KNBC, CHED2, BTR1, NABC1, CDPD, SGLT1, SGLT2, NIS, CHT 1, NET, DAT, GLYT2, CRTR, BOAT 1, SIT1, XT3, y+LAT 1, BAT1, NHERF1, NHE6, ASBT, DMT1, DCT1, NRAMP2, NKCC2, NCC, KCC3, NACT, MCT1, MCT8, MCT12, SLD, VGLUT3, THTR1, THTR2, PIT2, GLVR2, OCTN2, URAT1, NCKX1, NCKX5, CIC, PiC, ANT1, ORNT1, AGC1, ARALAR, Citrin, STLN2, aralar2, TPC, MUP1, MCPHA, CACT, GC1, PHC, DTD, CLD, DRA, PDS, Prestin, TAT1, FATP4, ENT3, ZnT2, ZnT10, AT1, NPT2A, NPT2B, HHRH, CST, CDG2F, UGAT, UGTL, UGALT, UGT1, UGT2, FUCT1, CDG2C, NST, PAT2, G6PT1, SPX4, ZIP4, LIV4, ZIP13, LZT-Hs9, FPN1, MTP1, IREG1, RHAG, AIM1, PCFT, FLVCR1, FLVCR2, RFT1, RFT2, RFT3, OATP1 B1, OATP1 B3, OATP2A1; structural proteins including but not limited to tubulin, heat shock protein, Microtubule-stabilizing proteins, Oncoprotein 18, stathmin, kinesin-8 and kinesin-14 family, Kip3, Kif18A; proteases including but not limited ADAM (a disintegrin and metalloprotease) family; Other molecule targets in signal transductions include but are not limited to: Cell division cycle 25 homolog A (CDC25A), forkhead box 03 (forkhead box 03), nuclear factor of kappa light polypeptide gene enhancer in B-cells inhibitor, alpha (NFKBIA), nuclear factor (erythroid-derived 2)-like 2 (NFE2L2), Natriuretic peptide receptor A (NPR1), Tumor necrosis factor receptor superfamily, member 11 a (TNFRSF11A), v-rel reticuloendotheliosis viral oncogene homolog A (avian) (RELA), Sterol regulatory element binding transcription factor 2 (SREBF2), CREB regulated transcription coactivator 1 (CRTC1), CREB regulated transcription coactivator 2 (CRTC2), X-box binding protein 1 (XBP1), Catenin (cadherin-associated protein), beta 1 (CTNNB1), and combinations thereof.

Examples of known biologics include, but are not limited to: Abbosynagis, Abegrin, Actemra, AFP-Cide, Antova, Arzerra, Aurexis, Avastin, Benlysta, Bexxar, Blontress, Bosatria, Campath, CEA-Cide, CEA-Scan, Cimzia, Cyramza, Ektomab, Erbitux, FibriScint, Gazyva, Flerceptin, hPAM4-Cide, FlumaSPECT, HuMax-CD4, HuMax-EGFr, Humira, HuZAF, Hybri-ceaker, Ilaris, Indimacis-125, Kadcyla, Lemtrada, LeukArrest, LeukoScan, Lucentis, Lymphomun, LymphoScan, LymphoStat-B, MabThera, Mycograb, Mylotarg, Myoscint, NeutroSpec, Numax, Nuvion, Omnitarg, Opdivo, Orthoclone OKT3, OvaRex, Panorex, Prolia, Prostascint, Raptiva, Remicade, Removab, Rencarex, ReoPro, Rexomun, Rituxan, RoActemra, Scintimun, Simponi, Simulect, Soliris, Stelara, Synagis, Tactress, Theracim, Theragyn, Theraloc, Tysabri, Vectibix, Verluma, Xolair, Yervoy, Zenapax, and Zevalin or combinations thereof.

Examples of known monoclonal antibodies include but are not limited to: 3F8, 8H9, Abagovomab, Abciximab, Abituzumab, Abrilumab, Actoxumab, Adalimumab, Adecatumumab, Aducanumab, Afasevikumab, Afelimomab, Afutuzumab, Alacizumab pegol, ALD518, ALD403, Alemtuzumab, Alirocumab, Altumomab pentetate, Amatuximab, AMG 334, Anatumomab mafenatox, Anetumab ravtansine, Anifrolumab, Anrukinzumab, Apolizumab, Arcitumomab, Ascrinvacumab, Aselizumab, Atezolizumab, Atinumab, Atlizumab, Atorolimumab, Avelumab, Bapineuzumab, Basiliximab, Bavituximab, Bectumomab, Begelomab, Belimumab, Benralizumab, Bertilimumab, Besilesomab, Bevacizumab, Bezlotoxumab, Biciromab, Bimagrumab, Bimekizumab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Canakinumab, Cantuzumab mertansine, Cantuzumab ravtansine, Caplacizumab, Capromab pendetide, Carlumab, Carotuximab, Catumaxomab, cBR96-doxorubicin immunoconjugate, Cedelizumab, Cergutuzumab amunaleukin, Certolizumab pegol, Cetuximab, Citatuzumab bogatox, Cixutumumab, Clazakizumab, Clenoliximab, Clivatuzumab tetraxetan, Codrituzumab, Coltuximab ravtansine, Conatumumab, Concizumab, CR6261, Crenezumab, Crotedumab, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dinutuximab, Diridavumab, Domagrozumab, Dorlimomab aritox, Drozitumab, Duligotumab, Dupilumab, Durvalumab, Dusigitumab, Ecromeximab, Eculizumab, Edobacomab, Edrecolomab, Efalizumab, Efungumab, Eldelumab, Elgemtumab, Elotuzumab, Elsilimomab, Emactuzumab, Emibetuzumab, Emicizumab, Enavatuzumab, Enfortumab vedotin, Enlimomab pegol, Enoblituzumab, Enokizumab, Enoticumab, Ensituximab, Epitumomab cituxetan, Epratuzumab, Erenumab, Erlizumab, Ertumaxomab, Etaracizumab, Etrolizumab, Evinacumab, Evolocumab, Exbivirumab, Fanolesomab, Faralimomab, Farletuzumab, Fasinumab, FBTA05, Felvizumab, Fezakinumab, Fibatuzumab, Ficlatuzumab, Figitumumab, Firivumab, Flanvotumab, Fletikumab, Fontolizumab, Foralumab, Foravirumab, Fresolimumab, Fulranumab, Futuximab, Galcanezumab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Idarucizumab, Igovomab, IMA-638, IMAB362, Imalumab, Imciromab, Imgatuzumab, Inclacumab, Indatuximab ravtansine, Indusatumab vedotin, Inebilizumab, Infliximab, Inolimomab, Inotuzumab ozogamicin, Intetumumab, Ipilimumab, Iratumumab, Isatuximab, Itolizumab, Ixekizumab, Keliximab, Labetuzumab, Lambrolizumab, Lampalizumab, Lanadelumab, Landogrozumab, Laprituximab emtansine, LBR-101/PF0442g7429, Lebrikizumab, Lemalesomab, Lendalizumab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, LY2951742, Mapatumumab, Margetuximab, Maslimomab, Matuzumab, Mavrilimumab, Mepolizumab, Metelimumab, Milatuzumab, Minretumomab, Mirvetuximab soravtansine, Mitumomab, Mogamulizumab, Monalizumab, Morolimumab, Motavizumab, Moxetumomab pasudotox, Muromonab-CD3, Nacolomab tafenatox, Nam ilumab, Naptumomab estafenatox, Naratuximab emtansine, Narnatumab, Natalizumab, Navicixizumab, Navivumab, Nebacumab, Necitumumab, Nemolizumab, Nerelimomab, Nesvacumab, Nimotuzumab, Nivolumab, Nofetumomab merpentan, Obiltoxaximab, Obinutuzumab, Ocaratuzumab, Ocrelizumab, Odulimomab, Ofatumumab, Olaratumab, Olokizumab, Omalizumab, Onartuzumab, Ontuxizumab, Opicinumab, Oportuzumab monatox, Oregovomab, Orticumab, Otelixizumab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Prezalizumab, Priliximab, Pritoxaximab, Pritumumab, PRO 140, Quilizumab, Racotumomab, Radretumab, Rafivirumab, Ralpancizumab, Ramucirumab, Ranibizumab, Raxibacumab, Refanezumab, Regavirumab, Reslizumab, Rilotumumab, Rinucumab, Risankizumab, Rituximab, Rivabazumab pegol, Robatumumab, Roledumab, Romosozumab, Rontalizumab, Rovalpituzumab tesirine, Rovelizumab, Ruplizumab, Sacituzumab govitecan, Samalizumab, Sapelizumab, Sarilumab, Satumomab pendetide, Secukinumab, Seribantumab, Setoxaximab, Sevirumab, SGN-CD19A, SGN-CD33A, Sibrotuzumab, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tamtuvetmab, Tanezumab, Taplitumomab paptox, Tarextumab, Tefibazumab, Telimomab aritox, Tenatumomab, Teneliximab, Teplizumab, Teprotumumab, Tesidolumab, Tetulomab, Tezepelumab, TGN1412, Ticilimumab, Tigatuzumab, Tildrakizumab, Timolumab, Tisotumab vedotin, TNX-650, Tocilizumab, Toralizumab, Tosatoxumab, Tositumomab, Tovetumab, Tralokinumab, Trastuzumab, Trastuzumab emtansine, TRBS07, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vandortuzumab vedotin, Vantictumab, Vanucizumab, Vapaliximab, Varlilumab, Vatelizumab, Vedolizumab, Veltuzumab, Vepalimomab, Vesencumab, Visilizumab, Vobarilizumab, Volociximab, Vorsetuzumab mafodotin, Votumumab, Xentuzumab, Zalutumumab, Zanolimumab, Zatuximab, Ziralimumab, and Zolimomab aritox or combinations thereof.

Examples of vaccines developed for viral diseases include but are not limited to: Hepatitis A vaccine, Hepatitis B vaccine, Hepatitis E vaccine, HPV vaccine, Influenza vaccine, Japanese encephalitis vaccine, MMR vaccine, MMRV vaccine, Polio vaccine, Rabies vaccine, Rotavirus vaccine, Varicella vaccine, Shingles vaccine, Smallpox vaccine, Yellow Fever vaccine, Adenovirus vaccine, Coxsackie B virus vaccine, Cytomegalovirus vaccine, Dengue vaccine for humans, Eastern Equine encephalitis virus vaccine for humans, Ebola vaccine, Enterovirus 71 vaccine, Epstein-Barr vaccine, Hepatitis C vaccine, HIV vaccine, HTLV-1 T-lymphotropic leukemia vaccine for humans, Marburg virus disease vaccine, Norovirus vaccine, Respiratory syncytial virus vaccine for humans, Severe acute respiratory syndrome (SARS) vaccine, West Nile virus vaccine for humans; Examples of bacterial diseases include but are not limited to: Anthrax vaccines, DPT vaccine, Q fever vaccine, Hib vaccine, Tuberculosis (BCG) vaccine, Meningococcal vaccine, Typhoid vaccine, Pneumococcal conjugate vaccine, Pneumococcal polysaccharide vaccine, Cholera vaccine, Caries vaccine, Ehrlichiosis vaccine, Leprosy vaccine, Lyme disease vaccine, Staphylococcus aureus vaccine, Streptococcus pyogenes vaccine, Syphilis vaccine, Tularemia vaccine, Yersinia pestis vaccine; Examples of parasitic diseases include but are not limited to: Malaria vaccine, Schistosomiasis vaccine, Chagas disease vaccine, Hookworm vaccine, Onchocerciasis river blindness vaccine for humans, Trypanosomiasis vaccine, Visceral leishmaniasis vaccine; Examples of non-infectious diseases include but are not limited to: Alzheimer's disease amyloid protein vaccine, Breast cancer vaccine, Ovarian cancer vaccine, Prostate cancer vaccine, Talimogene laherparepvec (T-VEC); also vaccines including but not limited to the following trade names: ACAM2000, ActHIB, Adacel, Afluria, AFLURIA QUADRIVALENT, Agriflu, BCG Vaccine, BEXSERO, Biothrax, Boostrix, Cervarix, Comvax, DAPTACEL, DECAVAC, Engerix-B, FLUAD, Fluarix, Fluarix Quadrivalent, Flublok, Flucelvax, Flucelvax Quadrivalent, FluLaval, FluMist, FluMist Quadrivalent, Fluvirin, Fluzone Quadrivalent, Fluzone, Fluzone High-Dose and Fluzone Intradermal, Gardasil, Gardasil 9, Havrix, Hiberix, Imovax, Infanrix, IPOL, Ixiaro, JE-Vax, KINRIX, Menactra, MenHibrix, Menomune-A/C/Y/W-135, Menveo, M-M-R II, M-M-Vax, Pediarix, PedvaxHIB, Pentacel, Pneumovax 23, Poliovax, Prevnar, Prevnar 13, ProQuad, Quadracel, Quadrivalent, RabAvert, Recombivax HB, ROTARIX, RotaTeq, TENIVAC, TICE BCG, Tripedia, TRUMENBA, Twinrix, TYPHIM Vi, VAQTA, Varivax, Vaxchora, Vivotif, YF-Vax, Zostavax, and combinations thereof.

Examples of injectable drugs include but are not limited to: Ablavar (Gadofosveset Trisodium Injection), Abarelix Depot, Abobotulinumtoxin A Injection (Dysport), ABT-263, ABT-869, ABX-EFG, Accretropin (Somatropin Injection), Acetadote (Acetylcysteine Injection), Acetazolamide Injection (Acetazolamide Injection), Acetylcysteine Injection (Acetadote), Actemra (Tocilizumab Injection), Acthrel (Corticorelin Ovine Triflutate for Injection), Actummune, Activase, Acyclovir for Injection (Zovirax Injection), Adacel, Adalimumab, Adenoscan (Adenosine Injection), Adenosine Injection (Adenoscan), Adrenaclick, AdreView (lobenguane 1123 Injection for Intravenous Use), Afluria, Ak-Fluor (Fluorescein Injection), Aldurazyme (Laronidase), Alglucerase Injection (Ceredase), Alkeran Injection (Melphalan Hcl Injection), Allopurinol Sodium for Injection (Aloprim), Aloprim (Allopurinol Sodium for Injection), Alprostadil, Alsuma (Sumatriptan Injection), ALTU-238, Amino Acid Injections, Aminosyn, Apidra, Apremilast, Alprostadil Dual Chamber System for Injection (Caverject Impulse), AMG 009, AMG 076, AMG 102, AMG 108, AMG 114, AMG 162, AMG 220, AMG 221, AMG 222, AMG 223, AMG 317, AMG 379, AMG 386, AMG 403, AMG 477, AMG 479, AMG 517, AMG 531, AMG 557, AMG 623, AMG 655, AMG 706, AMG 714, AMG 745, AMG 785, AMG 811, AMG 827, AMG 837, AMG 853, AMG 951, Amiodarone HCl Injection (Amiodarone HCl Injection), Amobarbital Sodium Injection (Amytal Sodium), Amytal Sodium (Amobarbital Sodium Injection), Anakinra, Anti-Abeta, Anti-Beta7, Anti-Beta20, Anti-CD4, Anti-CD20, Anti-CD40, Anti-IFNalpha, Anti-IL13, Anti-OX4OL, Anti-oxLDS, Anti-NGF, Anti-NRP1, Arixtra, Amphadase (Flyaluronidase Inj), Ammonul (Sodium Phenylacetate and Sodium Benzoate Injection), Anaprox, Anzemet Injection (Dolasetron Mesylate Injection), Apidra (Insulin Glulisine [rDNA origin] Inj), Apomab, Aranesp (darbepoetin alfa), Argatroban (Argatroban Injection), Arginine Flydrochloride Injection (R-Gene 10, Aristocort, Aristospan, Arsenic Trioxide Injection (Trisenox), Articane HCl and Epinephrine Injection (Septocaine), Arzerra (Ofatumumab Injection), Asclera (Polidocanol Injection), Ataluren, Ataluren-DMD, Atenolol Inj (Tenormin I.V. Injection), Atracurium Besylate Injection (Atracurium Besylate Injection), Avastin, Azactam Injection (Aztreonam Injection), Azithromycin (Zithromax Injection), Aztreonam Injection (Azactam Injection), Baclofen Injection (Lioresal Intrathecal), Bacteriostatic Water (Bacteriostatic Water for Injection), Baclofen Injection (Lioresal Intrathecal), Bal in Oil Ampules (Dimercarprol Injection), BayHepB, BayTet, Benadryl, Bendamustine Hydrochloride Injection (Treanda), Benztropine Mesylate Injection (Cogentin), Betamethasone Injectable Suspension (Celestone Soluspan), Bexxar, Bicillin C-R 900/300 (Penicillin G Benzathine and Penicillin G Procaine Injection), Blenoxane (Bleomycin Sulfate Injection), Bleomycin Sulfate Injection (Blenoxane), Boniva Injection (Ibandronate Sodium Injection), Botox Cosmetic (OnabotulinumtoxinA for Injection), BR3-FC, Bravelle (Urofollitropin Injection), Bretylium (Bretylium Tosylate Injection), Brevital Sodium (Methohexital Sodium for Injection), Brethine, Briobacept, BTT-1023, Bupivacaine HCl, Byetta, Ca-DTPA (Pentetate Calcium Trisodium Inj), Cabazitaxel Injection (Jevtana), Caffeine Alkaloid (Caffeine and Sodium Benzoate Injection), Calcijex Injection (Calcitrol), Calcitrol (Calcijex Injection), Calcium Chloride (Calcium Chloride Injection 10%), Calcium Disodium Versenate (Edetate Calcium Disodium Injection), Campath (Altemtuzumab), Camptosar Injection (Irinotecan Hydrochloride), Canakinumab Injection (Ilaris), Capastat Sulfate (Capreomycin for Injection), Capreomycin for Injection (Capastat Sulfate), Cardiolite (Prep kit for Technetium Tc99 Sestamibi for Injection), Carticel, Cathflo, Cefazolin and Dextrose for Injection (Cefazolin Injection), Cefepime Hydrochloride, Cefotaxime, Ceftriaxone, Cerezyme, Carnitor Injection, Caverject, Celestone Soluspan, Celsior, Cerebyx (Fosphenytoin Sodium Injection), Ceredase (Alglucerase Injection), Ceretec (Technetium Tc99m Exametazime Injection), Certolizumab, CF-101, Chloramphenicol Sodium Succinate (Chloramphenicol Sodium Succinate Injection), Chloramphenicol Sodium Succinate Injection (Chloramphenicol Sodium Succinate), Cholestagel (Colesevelam HCL), Choriogonadotropin Alfa Injection (Ovidrel), Cimzia, Cisplatin (Cisplatin Injection), Clolar (Clofarabine Injection), Clomiphine Citrate, Clonidine Injection (Duraclon), Cogentin (Benztropine Mesylate Injection), Colistimethate Injection (Coly-Mycin M), Coly-Mycin M (Colistimethate Injection), Compath, Conivaptan Hcl Injection (Vaprisol), Conjugated Estrogens for Injection (Premarin Injection), Copaxone, Corticorelin Ovine Triflutate for Injection (Acthrel), Corvert (Ibutilide Fumarate Injection), Cubicin (Daptomycin Injection), CF-101, Cyanokit (Hydroxocobalamin for Injection), Cytarabine Liposome Injection (DepoCyt), Cyanocobalamin, Cytovene (ganciclovir), D.H.E. 45, Dacetuzumab, Dacogen (Decitabine Injection), Dalteparin, Dantrium IV (Dantrolene Sodium for Injection), Dantrolene Sodium for Injection (Dantrium IV), Daptomycin Injection (Cubicin), Darbepoietin Alfa, DDAVP Injection (Desmopressin Acetate Injection), Decavax, Decitabine Injection (Dacogen), Dehydrated Alcohol (Dehydrated Alcohol Injection), Denosumab Injection (Prolia), Delatestryl, Delestrogen, Delteparin Sodium, Depacon (Valproate Sodium Injection), Depo Medrol (Methylprednisolone Acetate Injectable Suspension), DepoCyt (Cytarabine Liposome Injection), DepoDur (Morphine Sulfate XR Liposome Injection), Desmopressin Acetate Injection (DDAVP Injection), Depo-Estradiol, Depo-Provera 104 mg/mL, Depo-Provera 150 mg/mL, Depo-Testosterone, Dexrazoxane for Injection, Intravenous Infusion Only (Totect), Dextrose/Electrolytes, Dextrose and Sodium Chloride Inj (Dextrose 5% in 0.9% Sodium Chloride), Dextrose, Diazepam Injection (Diazepam Injection), Digoxin Injection (Lanoxin Injection), Dilaudid-HP (Hydromorphone Hydrochloride Injection), Dimercarprol Injection (Bal in Oil Ampules), Diphenhydramine Injection (Benadryl Injection), Dipyridamole Injection (Dipyridamole Injection), DMOAD, Docetaxel for Injection (Taxotere), Dolasetron Mesylate Injection (Anzemet Injection), Doribax (Doripenem for Injection), Doripenem for Injection (Doribax), Doxercalciferol Injection (Hectorol Injection), Doxil (Doxorubicin Hcl Liposome Injection), Doxorubicin Hcl Liposome Injection (Doxil), Duraclon (Clonidine Injection), Duramorph (Morphine Injection), Dysport (Abobotulinumtoxin A Injection), Ecallantide Injection (Kalbitor), EC-Naprosyn (naproxen), Edetate Calcium Disodium Injection (Calcium Disodium Versenate), Edex (Alprostadil for Injection), Engerix, Edrophonium Injection (Enlon), Eliglustat Tartate, Eloxatin (Oxaliplatin Injection), Emend Injection (Fosaprepitant Dimeglumine Injection), Enalaprilat Injection (Enalaprilat Injection), Enlon (Edrophonium Injection), Enoxaparin Sodium Injection (Lovenox), Eovist (Gadoxetate Disodium Injection), Enbrel (etanercept), Enoxaparin, Epicel, Epinepherine, Epipen, Epipen Jr., Epratuzumab, Erbitux, Ertapenem Injection (Invanz), Erythropoieten, Essential Amino Acid Injection (Nephramine), Estradiol Cypionate, Estradiol Valerate, Etanercept, Exenatide Injection (Byetta), Evlotra, Fabrazyme (Adalsidase beta), Famotidine Injection, FDG (Fludeoxyglucose F 18 Injection), Feraheme (Ferumoxytol Injection), Feridex I.V. (Ferumoxides Injectable Solution), Fertinex, Ferumoxides Injectable Solution (Feridex I.V.), Ferumoxytol Injection (Feraheme), Flagyl Injection (Metronidazole Injection), Fluarix, Fludara (Fludarabine Phosphate), Fludeoxyglucose F 18 Injection (FDG), Fluorescein Injection (Ak-Fluor), Follistim AQ Cartridge (Follitropin Beta Injection), Follitropin Alfa Injection (Gonal-f RFF), Follitropin Beta Injection (Follistim AQ Cartridge), Folotyn (Pralatrexate Solution for Intravenous Injection), Fondaparinux, Forteo (Teriparatide (rDNA origin) Injection), Fostamatinib, Fosaprepitant Dimeglumine Injection (Emend Injection), Foscarnet Sodium Injection (Foscavir), Foscavir (Foscarnet Sodium Injection), Fosphenytoin Sodium Injection (Cerebyx), Fospropofol Disodium Injection (Lusedra), Fragmin, Fuzeon (enfuvirtide), GA101, Gadobenate Dimeglumine Injection (Multihance), Gadofosveset Trisodium Injection (Ablavar), Gadoteridol Injection Solution (ProFlance), Gadoversetamide Injection (OptiMARK), Gadoxetate Disodium Injection (Eovist), Ganirelix (Ganirelix Acetate Injection), Gardasil, GC1008, GDFD, Gemtuzumab Ozogamicin for Injection (Mylotarg), Genotropin, Gentamicin Injection, GENZ-112638, Golimumab Injection (Simponi Injection), Gonal-f RFF (Follitropin Alfa Injection), Granisetron Flydrochloride (Kytril Injection), Gentamicin Sulfate, Glatiramer Acetate, Glucagen, Glucagon, HAE1, Haldol (Flaloperidol Injection), Flavrix, Flectorol Injection (Doxercalciferol Injection), Fledgehog Pathway Inhibitor, Fleparin, Flerceptin, hG-CSF, Flumalog, Fluman Growth Hormone, Humatrope, HuMax, Humegon, Humira, Humulin, Ibandronate Sodium Injection (Boniva Injection), Ibuprofen Lysine Injection (NeoProfen), Ibutilide Fumarate Injection (Corvert), Idamycin PFS (Idarubicin Hydrochloride Injection), Idarubicin Hydrochloride Injection (Idamycin PFS), Ilaris (Canakinumab Injection), Imipenem and Cilastatin for Injection (Primaxin I.V.), Imitrex, Incobotulinumtoxin A for Injection (Xeomin), Increlex (Mecasermin [rDNA origin] Injection), Indocin IV (Indomethacin Inj), Indomethacin Inj (Indocin IV), Infanrix, Innohep, Insulin, Insulin Aspart [rDNA origin] Inj (NovoLog), Insulin Glargine [rDNA origin] Injection (Lantus), Insulin Glulisine [rDNA origin] Inj (Apidra), Interferon alfa-2b, Recombinant for Injection (Intron A), Intron A (Interferon alfa-2b, Recombinant for Injection), Invanz (Ertapenem Injection), Invega Sustenna (Paliperidone Palmitate Extended-Release Injectable Suspension), Invirase (saquinavir mesylate), lobenguane 1123 Injection for Intravenous Use (AdreView), Iopromide Injection (Ultravist), Ioversol Injection (Optiray Injection), Iplex (Mecasermin Rinfabate [rDNA origin] Injection), Iprivask, Irinotecan Hydrochloride (Camptosar Injection), Iron Sucrose Injection (Venofer), Istodax (Romidepsin for Injection), Itraconazole Injection (Sporanox Injection), Jevtana (Cabazitaxel Injection), Jonexa, Kalbitor (Ecallantide Injection), KCL in D5NS (Potassium Chloride in 5% Dextrose and Sodium Chloride Injection), KCL in D5W, KCL in NS, Kenalog 10 Injection (Triamcinolone Acetonide Injectable Suspension), Kepivance (Palifermin), Keppra Injection (Levetiracetam), Keratinocyte, KFG, Kinase Inhibitor, Kineret (Anakinra), Kinlytic (Urokinase Injection), Kinrix, Klonopin (clonazepam), Kytril Injection (Granisetron Hydrochloride), lacosamide Tablet and Injection (Vimpat), Lactated Ringer's, Lanoxin Injection (Digoxin Injection), Lansoprazole for Injection (Prevacid I.V.), Lantus, Leucovorin Calcium (Leucovorin Calcium Injection), Lente (L), Leptin, Levemir, Leukine Sargramostim, Leuprolide Acetate, Levothyroxine, Levetiracetam (Keppra Injection), Lovenox, Levocarnitine Injection (Carnitor Injection), Lexiscan (Regadenoson Injection), Lioresal Intrathecal (Baclofen Injection), Liraglutide [rDNA] Injection (Victoza), Lovenox (Enoxaparin Sodium Injection), Lucentis (Ranibizumab Injection), Lumizyme, Lupron (Leuprolide Acetate Injection), Lusedra (Fospropofol Disodium Injection), Maci, Magnesium Sulfate (Magnesium Sulfate Injection), Mannitol Injection (Mannitol IV), Marcaine (Bupivacaine Hydrochloride and Epinephrine Injection), Maxipime (Cefepime Hydrochloride for Injection), MDP Multidose Kit of Technetium Injection (Technetium Tc99m Medronate Injection), Mecasermin [rDNA origin] Injection (Increlex), Mecasermin Rinfabate [rDNA origin] Injection (Iplex), Melphalan Hcl Injection (Alkeran Injection), Methotrexate, Menactra, Menopur (Menotropins Injection), Menotropins for Injection (Repronex), Methohexital Sodium for Injection (Brevital Sodium), Methyldopate Hydrochloride Injection, Solution (Methyldopate Hcl), Methylene Blue (Methylene Blue Injection), Methylprednisolone Acetate Injectable Suspension (Depo Medrol), MetMab, Metoclopramide Injection (Reglan Injection), Metrodin (Urofollitropin for Injection), Metronidazole Injection (Flagyl Injection), Miacalcin, Midazolam (Midazolam Injection), Mimpara (Cinacalet), Minocin Injection (Minocycline Inj), Minocycline Inj (Minocin Injection), Mipomersen, Mitoxantrone for Injection Concentrate (Novantrone), Morphine Injection (Duramorph), Morphine Sulfate XR Liposome Injection (DepoDur), Morrhuate Sodium (Morrhuate Sodium Injection), Motesanib, Mozobil (Plerixafor Injection), Multihance (Gadobenate Dimeglumine Injection), Multiple Electrolytes and Dextrose Injection, Multiple Electrolytes Injection, Mylotarg (Gemtuzumab Ozogamicin for Injection), Myozyme (Alglucosidase alfa), Nafcillin Injection (Nafcillin Sodium), Nafcillin Sodium (Nafcillin Injection), Naltrexone XR Inj (Vivitrol), Naprosyn (naproxen), NeoProfen (Ibuprofen Lysine Injection), Nandrol Decanoate, Neostigmine Methylsulfate (Neostigmine Methylsulfate Injection), NEO-GAA, NeoTect (Technetium To 99m Depreotide Injection), Nephramine (Essential Amino Acid Injection), Neulasta (pegfilgrastim), Neupogen (Filgrastim), Novolin, Novolog, NeoRecormon, Neutrexin (Trimetrexate Glucuronate Inj), NPH (N), Nexterone (Amiodarone HCl Injection), Norditropin (Somatropin Injection), Normal Saline (Sodium Chloride Injection), Novantrone (Mitoxantrone for Injection Concentrate), Novolin 70/30 Innolet (70% NPH, Human Insulin Isophane Suspension and 30% Regular, Human Insulin Injection), NovoLog (Insulin Aspart [rDNA origin] Inj), Nplate (romiplostim), Nutropin (Somatropin (rDNA origin) for Inj), Nutropin AQ, Nutropin Depot (Somatropin (rDNA origin) for Inj), Octreotide Acetate Injection (Sandostatin LAR), Ocrelizumab, Ofatumumab Injection (Arzerra), Olanzapine Extended Release Injectable Suspension (Zyprexa Relprevv), Omnitarg, Omnitrope (Somatropin [rDNA origin] Injection), Ondansetron Hydrochloride Injection (Zofran Injection), OptiMARK (Gadoversetamide Injection), Optiray Injection (Ioversol Injection), Orencia, Osmitrol Injection in Aviva (Mannitol Injection in Aviva Plastic Vessel 250), Osmitrol Injection in Viaflex (Mannitol Injection in Viaflex Plastic Vessel 250), Osteoprotegrin, Ovidrel (Choriogonadotropin Alfa Injection), Oxacillin (Oxacillin for Injection), Oxaliplatin Injection (Eloxatin), Oxytocin Injection (Pitocin), Paliperidone Palmitate Extended-Release Injectable Suspension (Invega Sustenna), Pamidronate Disodium Injection (Pamidronate Disodium Injection), Panitumumab Injection for Intravenous Use (Vectibix), Papaverine Hydrochloride Injection (Papaverine Injection), Papaverine Injection (Papaverine Hydrochloride Injection), Parathyroid Hormone, Paricalcitol Injection Fliptop Vial (Zemplar Injection), PARP Inhibitor, Pediarix, PEGIntron, Peginterferon, Pegfilgrastim, Penicillin G Benzathine and Penicillin G Procaine, Pentetate Calcium Trisodium Inj (Ca-DTPA), Pentetate Zinc Trisodium Injection (Zn-DTPA), Pepcid Injection (Famotidine Injection), Pergonal, Pertuzumab, Phentolamine Mesylate (Phentolamine Mesylate for Injection), Physostigmine Salicylate (Physostigmine Salicylate (injection)), Physostigmine Salicylate (injection) (Physostigmine Salicylate), Piperacillin and Tazobactam Injection (Zosyn), Pitocin (Oxytocin Injection), Plasma-Lyte 148 (Multiple Electrolytes Inj), Plasma-Lyte 56 and Dextrose (Multiple Electrolytes and Dextrose Injection in Viaflex, Plastic Vessel 250), PlasmaLyte, Plerixafor Injection (Mozobil), Polidocanol Injection (Asclera), Potassium Chloride, Pralatrexate Solution for Intravenous Injection (Folotyn), Pram lintide Acetate Injection (Symlin), Premarin Injection (Conjugated Estrogens for Injection), Prep kit for Technetium Tc99 Sestamibi for Injection (Cardiolite), Prevacid I.V. (Lansoprazole for Injection), Primaxin I.V. (Imipenem and Cilastatin for Injection), Prochymal, Procrit, Progesterone, ProHance (Gadoteridol Injection Solution), Prolia (Denosumab Injection), Promethazine HCl Injection (Promethazine Hydrochloride Injection), Propranolol Hydrochloride Injection (Propranolol Hydrochloride Injection), Quinidine Gluconate Injection (Quinidine Injection), Quinidine Injection (Quinidine Gluconate Injection), R-Gene 10 (Arginine Hydrochloride Injection), Ranibizumab Injection (Lucentis), Ranitidine Hydrochloride Injection (Zantac Injection), Raptiva, Reclast (Zoledronic Acid Injection), Recombivarix HB, Regadenoson Injection (Lexiscan), Reglan Injection (Metoclopramide Injection), Remicade, Renagel, Renvela (Sevelamer Carbonate), Repronex (Menotropins for Injection), Retrovir IV (Zidovudine Injection), rhApo2L/TRAIL, Ringer's and 5% Dextrose Injection (Ringers in Dextrose), Ringer's Injection (Ringers Injection), Rituxan, Rituximab, Rocephin (ceftriaxone), Rocuronium Bromide Injection (Zemuron), Roferon-A (interferon alfa-2a), Romazicon (flumazenil), Romidepsin for Injection (Istodax), Saizen (Somatropin Injection), Sandostatin LAR (Octreotide Acetate Injection), Sclerostin Ab, Sensipar (cinacalcet), Sensorcaine (Bupivacaine HCl Injections), Septocaine (Articane HCl and Epinephrine Injection), Serostim LQ (Somatropin (rDNA origin) Injection), Simponi Injection (Golimumab Injection), Sodium Acetate (Sodium Acetate Injection), Sodium Bicarbonate (Sodium Bicarbonate 5% Injection), Sodium Lactate (Sodium Lactate Injection in AVIVA), Sodium Phenylacetate and Sodium Benzoate Injection (Ammonul), Somatropin (rDNA origin) for Inj (Nutropin), Sporanox Injection (Itraconazole Injection), Stelara Injection (Ustekinumab), Stemgen, Sufenta (Sufentanil Citrate Injection), Sufentanil Citrate Injection (Sufenta), Sumavel, Sumatriptan Injection (Alsuma), Symlin, Symlin Pen, Systemic Hedgehog Antagonist, Synvisc-One (Hylan G-F 20 Single Intra-articular Injection), Tarceva, Taxotere (Docetaxel for Injection), Technetium Tc 99m, Telavancin for Injection (Vibativ), Temsirolimus Injection (Torisel), Tenorm in I.V. Injection (Atenolol Inj), Teriparatide (rDNA origin) Injection (Forteo), Testosterone Cypionate, Testosterone Enanthate, Testosterone Propionate, Tev-Tropin (Somatropin, rDNA Origin, for Injection), tgAAC94, Thallous Chloride, Theophylline, Thiotepa (Thiotepa Injection), Thymoglobulin (Anti-Thymocyte Globulin (Rabbit), Thyrogen (Thyrotropin Alfa for Injection), Ticarcillin Disodium and Clavulanate Potassium Galaxy (Timentin Injection), Tigan Injection (Trimethobenzamide Hydrochloride Injectable), Timentin Injection (Ticarcillin Disodium and Clavulanate Potassium Galaxy), TNKase, Tobramycin Injection (Tobramycin Injection), Tocilizumab Injection (Actemra), Torisel (Temsirolimus Injection), Totect (Dexrazoxane for Injection, Intravenous Infusion Only), Trastuzumab-DM1, Travasol (Amino Acids (Injection)), Treanda (Bendamustine Hydrochloride Injection), Trelstar (Triptorelin Pamoate for Injectable Suspension), Triamcinolone Acetonide, Triamcinolone Diacetate, Triamcinolone Hexacetonide Injectable Suspension (Aristospan Injection 20 mg), Triesence (Triamcinolone Acetonide Injectable Suspension), Trimethobenzamide Hydrochloride Injectable (Tigan Injection), Trimetrexate Glucuronate Inj (Neutrexin), Triptorelin Pamoate for Injectable Suspension (Trelstar), Twinject, Trivaris (Triamcinolone Acetonide Injectable Suspension), Trisenox (Arsenic Trioxide Injection), Twinrix, Typhoid Vi, Ultravist (Iopromide Injection), Urofollitropin for Injection (Metrodin), Urokinase Injection (Kinlytic), Ustekinumab (Stelara Injection), Ultralente (U), Valium (diazepam), Valproate Sodium Injection (Depacon), Valtropin (Somatropin Injection), Vancomycin Hydrochloride (Vancomycin Hydrochloride Injection), Vancomycin Hydrochloride Injection (Vancomycin Hydrochloride), Vaprisol (Conivaptan Hcl Injection), VAQTA, Vasovist (Gadofosveset Trisodium Injection for Intravenous Use), Vectibix (Panitumumab Injection for Intravenous Use), Venofer (Iron Sucrose Injection), Verteporfin Inj (Visudyne), Vibativ (Telavancin for Injection), Victoza (Liraglutide [rDNA] Injection), Vimpat (lacosamide Tablet and Injection), Vinblastine Sulfate (Vinblastine Sulfate Injection), Vincasar PFS (Vincristine Sulfate Injection), Victoza, Vincristine Sulfate (Vincristine Sulfate Injection), Visudyne (Verteporfin Inj), Vitamin B-12, Vivitrol (Naltrexone XR Inj), Voluven (Hydroxyethyl Starch in Sodium Chloride Injection), Xeloda, Xenical (orlistat), Xeomin (Incobotulinumtoxin A for Injection), Xolair, Zantac Injection (Ranitidine Hydrochloride Injection), Zemplar Injection (Paricalcitol Injection Fliptop Vial), Zemuron (Rocuronium Bromide Injection), Zenapax (daclizumab), Zevalin, Zidovudine Injection (Retrovir IV), Zithromax Injection (Azithromycin), Zn-DTPA (Pentetate Zinc Trisodium Injection), Zofran Injection (Ondansetron Hydrochloride Injection), Zingo, Zoledronic Acid for Inj (Zometa), Zoledronic Acid Injection (Reclast), Zometa (Zoledronic Acid for Inj), Zosyn (Piperacillin and Tazobactam Injection), Zyprexa Relprevv (Olanzapine Extended Release Injectable Suspension) and combinations thereof.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

### PROPHETIC EXAMPLES

### Prophetic Example A: Impact of Surfactants on Fatty Acid Particle Formation

Sample Preparation: Samples containing an enzyme having lipase activity (e.g., rabbit liver esterase, pancreatic lipase) and a buffer (e.g., 20 mM histidine chloride buffer, pH 5.5) will be prepared in 10 cc vials, with each sample having a volume of approximately 6 mL. Except for the control sample, the samples will contain various types and concentrations of polysorbate surfactants, including polysorbate 20 (PS20), polysorbate 40 (PS40), polysorbate 60 (PS60), and polysorbate 80 (PS80), as shown in Table 1 below.

TABLE 1

Surfactant (wt. %)

| Sample | PS20 | PS40 | PS60 | PS80 |
|---|---|---|---|---|
| Control | 0.00 | 0.00 | 0.00 | 0.00 |
| 20-A | 0.01 | 0.00 | 0.00 | 0.00 |
| 20-B | 0.02 | 0.00 | 0.00 | 0.00 |
| 20-C | 0.04 | 0.00 | 0.00 | 0.00 |
| 20-D | 0.08 | 0.00 | 0.00 | 0.00 |
| 20-E | 0.10 | 0.00 | 0.00 | 0.00 |
| 20-F | 0.15 | 0.00 | 0.00 | 0.00 |
| 20-G | 0.30 | 0.00 | 0.00 | 0.00 |
| 40-A | 0.00 | 0.01 | 0.00 | 0.00 |
| 40-B | 0.00 | 0.02 | 0.00 | 0.00 |
| 40-C | 0.00 | 0.04 | 0.00 | 0.00 |
| 40-D | 0.00 | 0.08 | 0.00 | 0.00 |
| 40-E | 0.00 | 0.10 | 0.00 | 0.00 |
| 40-F | 0.00 | 0.15 | 0.00 | 0.00 |
| 40-G | 0.00 | 0.30 | 0.00 | 0.00 |
| 60-A | 0.00 | 0.00 | 0.01 | 0.00 |
| 60-B | 0.00 | 0.00 | 0.02 | 0.00 |
| 60-C | 0.00 | 0.00 | 0.04 | 0.00 |
| 60-D | 0.00 | 0.00 | 0.08 | 0.00 |
| 60-E | 0.00 | 0.00 | 0.10 | 0.00 |
| 60-F | 0.00 | 0.00 | 0.15 | 0.00 |
| 60-G | 0.00 | 0.00 | 0.30 | 0.00 |
| 80-A | 0.00 | 0.00 | 0.00 | 0.01 |
| 80-B | 0.00 | 0.00 | 0.00 | 0.02 |
| 80-C | 0.00 | 0.00 | 0.00 | 0.04 |
| 80-D | 0.00 | 0.00 | 0.00 | 0.08 |
| 80-E | 0.00 | 0.00 | 0.00 | 0.10 |
| 80-F | 0.00 | 0.00 | 0.00 | 0.15 |
| 80-G | 0.00 | 0.00 | 0.00 | 0.30 |

Incubation: Each sample will be incubated at 40° C. for 2 months and then incubated to 5° C. for at least 24 hours.

Analysis: After the 24-hour incubation period, each sample will be analyzed for fatty acid particle formation at predetermined times, such as 0 hours, 1 day, 2 days, 4 days, 10 days, 30 days, and 60 days.

Each sample may be subjected to visual inspection in a light box, such as the Seidenader V90-T. This visual inspection may be performed at 5° C., at room temperature, or both.

Each sample may be subjected to micro-flow imaging (MFI). Each sample would remain at 5° C. until just before MFI analysis.

Each sample may also be subjected to high performance liquid chromatography (HPLC) to quantify any polysorbate surfactant that remains intact or any free fatty acids.

Prophetic Results: The present inventors believe that fatty acid particles will be visible in samples 20-A through 80-G after 60 days or less, more likely after 10 days or less. The present inventors also believe that fatty acid particles will be visible in samples having higher surfactant concentrations (e.g., 20-G, 40-G, 60-G, 80-G) before samples having lower surfactant concentrations (e.g., 20-A, 40-A, 60-A, 80-A). Without wishing to be bound by theory, the present inventors believe that fatty acid particles will not be visible in the control sample, even after 60 days.

### Prophetic Example B: Impact of Headspace on Agitation Induced Protein Aggregation

Sample Preparation: Samples containing a protein (e.g., BSA, hGH) in 150 mM NaCl and a buffer (e.g., 20 mM histidine chloride buffer, pH 6) will be prepared in 3 cc or larger vials. The samples will have different protein concentrations (e.g., 1, 10, 50, 150 mg/mL) and different PS20 concentrations (e.g., 0.0 wt. %, 0.01 wt. %, 0.02 wt. %, 0.04 wt. %, 0.08 wt. %, 0.10 wt. %, 0.15 wt. %, and 0.30 wt. %). Syringes of different volumes (e.g., 0.5 mL, 1 mL, and 3 mL) will be filled aseptically with appropriate amounts of each sample to achieve different headspace volumes (e.g., 0 mL, 0.05 mL, 0.1 mL, 0.2 mL, 0.5 mL, 1.0 mL). Then, each syringe will be capped and sealed.

Agitation: Each syringe and vial will be agitated back and forth using a suitable agitator for 24 hours at room temperature.

Analysis: The contents of each syringe and vial will be inspected for particles and opalescence. Each sample may be analyzed using a suitable spectrometer at 400 nm and 500 nm with 1 cm path length, with the samples being neat, pre- and post-0.22 um filtration. Each sample may also be analyzed using Size Exclusion Chromatography (SEC) for soluble aggregates (post-0.22 um filtration).

Prophetic Results: For all samples containing PS20, the present inventors believe that the samples will have a low surface tension at the air-water interface (due to the presence of PS20 at that interface) and will exhibit no changes following agitation. For samples in vials without PS20, on the other hand, the present inventors believe that the samples will have a higher surface tension and will exhibit increased opalescence and possibly increase soluble aggregates. However, the present inventors believe that such opalescence and aggregation will not occur in the prefilled syringes that have low headspace volume due to the higher surface tension at the air-water interface. This demonstrates that even samples without PS20 will exhibit no changes following agitation when headspace volume in a syringe is minimized.

1. A pre-filled injection device comprising:
a stopper;
a barrel;
a solid lubricant on at least one of the stopper and the barrel, the pre-filled injection device being free or substantially free of a liquid lubricant; and
a therapeutic formulation having lipase activity and comprising at least about 1 mg/ml of one or more active pharmacological agents,
wherein the therapeutic formulation is free or substantially free of fatty acid particles.

2. The pre-filled injection device of claim 1, wherein the active pharmacological agents comprise proteins, antibodies, cytokines, growth factors, coagulation factors, proteases, kinases, phosphatases, vaccines, peptides, small interfering RNAs (siRNAs), small interfering DNAs (siDNAs), messenger RNAs (mRNAs), messenger RNAs (mRNAs), aptamers, and/or any combination thereof.

3. The pre-filled injection device of claim 1, wherein the therapeutic formulation includes the one or more active pharmacological agents at a concentration from about 1 mg/ml to about 200 mg/ml, from about 10 mg/ml to about 200 mg/ml, from about 20 mg/ml to about 200 mg/ml, from about 40 mg/ml to about 200 mg/ml, from about 60 mg/ml to about 200 mg/ml, from about 80 mg/ml to about 200 mg/ml, from about 100 mg/ml to about 200 mg/ml, from about 120 mg/ml to about 200 mg/ml, and/or from about 150 mg/ml to about 200 mg/ml.

4. The pre-filled injection device of claim 1, wherein the therapeutic formulation is free or substantially free of a surfactant, and wherein the surfactant comprises at least one of polysorbate 20, polysorbate 40, polysorbate 60, polysorbate 80, and/or a combination thereof.

5. The pre-filled injection device of claim 4, wherein the therapeutic formulation includes the surfactant at a concentration from 0 wt. % to about 0.1 wt. %, from 0 wt. % to about 0.075 wt. %, from 0 wt. % to about 0.05 wt. %, from 0 wt. % to about 0.025 wt. %, from 0 wt. % to about 0.01 wt. %, from 0 wt. % to about 0.005 wt. %, and/or from 0 wt. % to about 0.001 wt. %.

6. The pre-filled injection device of claim 1, wherein the therapeutic formulation comprises a buffer having a pH from about 4.0 to about 9.5, from about 4.5 to about 9.0, from about 5.0 to about 8.5, from about 5.5 to about 8.0, from about 5.5 to about 7.5, from about 5.5 to about 7.0, or from about 5.5 to about 6.5.

7. The pre-filled injection device of claim 1, the therapeutic formulation comprises a sugar having a concentration from 0 wt. % to about 15 wt. %, from about 0.1 wt. % to about 15 wt. %, from about 1 wt. % to about 15 wt. %, from about 1.5 wt. % to about 10 wt. %, from about 2 wt. % to about 10 wt. %, from about 3 wt. % to about 10 wt. %, and/or from about 5 wt. % to about 10 wt. %.

8. The pre-filled injection device of claim 1, wherein the therapeutic formulation comprises a polyol having a concentration from 0 wt. % to about 5 wt. %, from about 0.1 wt. % to about 5 wt. %, from about 1 wt. % to about 5 wt. %, from about 1.5 wt. % to about 5 wt. %, from about 2 wt. % to about 5 wt. %, and/or from about 3 wt. % to about 5 wt. %.

9. The pre-filled injection device of claim 1, wherein the therapeutic formulation comprises an arginine salt having a concentration from 0 wt. % to about 5 wt. %, from about 0.1 wt. % to about 5 wt. %, from about 1 wt. % to about 5 wt. %, from about 1.5 wt. % to about 5 wt. %, from about 2 wt. % to about 5 wt. %, and/or from about 3 wt. % to about 5 wt. %.

10. The pre-filled injection device of claim 1, wherein the stopper and the barrel are free or substantially free of the liquid lubricant silicone.

11. The pre-filled injection device of claim 1, wherein the barrel is made of at least one of a glass material, a plastic material, a ceramic material, a metallic material or a combination thereof.

12. The pre-filled injection device of claim 1, wherein the solid lubricant is a low coefficient of friction polymer layer and the stopper comprises an elastomeric body and the low coefficient of friction polymer layer positioned on the elastomeric body.

13. The pre-filled injection device of claim 12, wherein the low coefficient of friction polymer layer comprises a fluoropolymer.

14. The pre-filled injection device of claim 13, wherein the fluoropolymer of the low coefficient of friction polymer layer is an expanded fluoropolymer.

15. The pre-filled injection device of claim 14, wherein the elastomeric body is at least partially imbibed into the expanded fluoropolymer of the low coefficient of friction polymer layer.

16. The pre-filled injection device of claim 13, wherein the fluoropolymer of the low coefficient of friction polymer layer is an expanded polytetrafluoroethylene (ePTFE).

17. The pre-filled injection device of claim 13, wherein the fluoropolymer of the low coefficient of friction polymer layer comprises a composite fluoropolymer film having a barrier layer and a porous layer, the barrier layer comprising at least one of polytetrafluoroethylene (PTFE), expanded polytetrafluoroethylene (ePTFE), densified ePTFE, fluorinated ethylene propylene (FEP), ethylene tetrafluoroethylene (ETFE), polyvinylfluoride, polyvinylidene fluoride, perfluoropropylvinylether, perfluoroalkoxy polymers, polyethylene, polypropylene, polylactic acid (PLA), poly(L-lactic acid) (PLLA), poly(D-lactic acid) (PDLA), poly (p-xylylene) (PPX), and copolymers and combinations thereof.

18. The pre-filled injection device of claim 1, wherein the therapeutic formulation in the pre-filled injection device includes fatty acid particles of a size greater than 25 microns from 0 to about 600 particles, from 0 to about 300 particles, from 0 to about 100 particles, from 0 to about 20 particles, from 0 to about 5 particles, from 0 to about 2 particles, or from 0 to about 1 particles.

19. The pre-filled injection device of claim 1, wherein the therapeutic formulation in the pre-filled injection device includes fatty acid particles of a size greater than 10 microns from 0 to about 6000 particles, from 0 to about 3000 particles, from 0 to about 1000 particles, from 0 to about 200 particles, from 0 to about 50 particles, from 0 to about 20 particles, and/or from 0 to about 10 particles.

20. The pre-filled injection device of claim 1, wherein the therapeutic formulation is free or substantially free of fatty acid particles when stored at 2° C. to 8° C. for at least about 1 year.

21. The pre-filled injection device of claim 20, wherein the therapeutic formulation is free or substantially free of fatty acid particles when stored at 2° C. to 8° C. for at least about 2 years.

22. The pre-filled injection device of claim 1, wherein the therapeutic formulation is free or substantially free of fatty acid particles after being subjected to accelerated aging, which involves heating the therapeutic formulation to 40° C. for 2 months and then cooling the therapeutic formulation to 5° C. for at least 24 hours.

23.-98. (canceled)
